(19) United States
(12) Patent Application Publication
(10) Pub. No.: US 2021/0259759 A1
(43) Pub. Date: Aug. 26, 2021

(54) ELECTROSURGICAL SYSTEM AND METHODS OF SWITCHING BETWEEN DISTINCT MODES AND POWER SETTINGS

(71) Applicant: Covidien LP, Mansfield, MA (US)

(72) Inventors: Jennifer R. Mchenry, Denver, CO (US); William D. Faulkner, Boulder, CO (US); James E. Dunning, Boulder, CO (US); Devon E. Scott-Drechsel, Superior, CO (US); Eric M. Westra, Loveland, CO (US); Donald L. Tonn, Superior, CO (US)

(21) Appl. No.: 17/179,579

(22) Filed: Feb. 19, 2021

Related U.S. Application Data
(60) Provisional application No. 62/978,949, filed on Feb. 20, 2020.

(51) Int. Cl. A61B 18/12

(52) U.S. Cl. CPC A61B 18/1206; A61B 2018/00922

(57) ABSTRACT

In accordance with aspects of the present disclosure, an adaptor assembly includes a plug, a selector assembly, and a connector. The plug is coupled to an electrosurgical generator and is configured to receive electrosurgical energy from the electrosurgical generator. The selector assembly includes a first position and a second position. The first position is configured to indicate a first setting of the electrosurgical generator. The second position is configured to indicate a second setting of the electrosurgical generator. The selector assembly, when actuated, indicates the selected position to the electrosurgical generator to initiate the corresponding setting. The connector is configured to couple to a surgical instrument and convey the electrosurgical energy to the surgical instrument. The electrosurgical energy, when based on the first setting, is different from the electrosurgical energy, when based on the second setting.

### CROSS-REFERENCE TO RELATED APPLICATIONS

This application claims the benefit of and priority to U.S. Provisional Patent Application No. 62/978,949, filed Feb. 20, 2020. The entire disclosures of the foregoing applications are incorporated by reference herein.

### FIELD

The present technology is generally related to an electrosurgical system and method, and more particularly, to an electrosurgical system and method with mode and power setting switching.

### BACKGROUND

Electrosurgical generators are employed in conjunction with an electrosurgical instrument to cut, resect, coagulate, desiccate and/or seal patient tissue. High frequency electrical energy, e.g., radio frequency (RF) energy, is produced by the electrosurgical generator and is applied to the tissue by an electrosurgical instrument. Electrosurgical instruments may include monopolar and/or bipolar configurations commonly used during electrosurgical procedures having active and/or return conductors or prongs. Typically, different waveforms of electrosurgical energy are used by electrosurgical instruments to achieve different surgical affects, e.g., cutting, resecting, coagulating, desiccating, sealing, etc. Different waveforms of electrosurgical energy are typically controlled by the electrosurgical generator and require manual input from the user on the electrosurgical generator to achieve a different waveform for the desired surgical affect.

### SUMMARY

The present disclosure generally relates to an adaptor assembly coupled to an electrosurgical generator that permits a clinician to adjust generator settings by interacting with the adaptor assembly rather than interacting with the generator.

In one aspect, the present disclosure provides an adaptor assembly that includes a plug, a selector assembly, and a connector. The plug is configured to couple to an electrosurgical generator and receive electrosurgical energy from the electrosurgical generator. The selector assembly includes a first position and a second position. The first position is configured to indicate a first setting of the electrosurgical generator. The second position is configured to indicate a second setting of the electrosurgical generator. The selector assembly, when actuated, indicates the selected position to the electrosurgical generator to initiate the corresponding setting. The connector is configured to couple to a surgical instrument and convey the electrosurgical energy to the surgical instrument. The electrosurgical energy, when based on the first setting, is different from the electrosurgical energy, when based on the second setting.

In aspects, the plug may further include a first prong, a second prong, a third prong, and an active prong. Each prong may be configured to be inserted into a corresponding receptacle of the electrosurgical generator.

In aspects, the adaptor assembly may further include an active conductor coupled to the active prong. The selector assembly may be actuated in the first position or the second position. The electrosurgical energy may be conveyed from the active prong through the active conductor to the surgical instrument.

In aspects, the adaptor assembly may further include a first conductor coupled to the first prong. The selector assembly may be actuated in the first position and the selector assembly may complete an electrical loop between the first conductor and the active conductor.

In aspects, the adaptor assembly may further include a second conductor coupled to the second prong. The selector assembly may be actuated in the second position and the selector assembly may complete an electrical loop between the second conductor and the active conductor.

In aspects, the adaptor assembly may further include an electrical circuit coupled to the third prong.

In aspects, the electrical circuit may be configured to deliver an output voltage indicative of an identity of the adaptor assembly when the adaptor assembly is coupled to the connector, where the output voltage is derived from at least a portion of the electrosurgical energy from the electrosurgical generator.

In aspects, the adaptor assembly may further include an active conductor, a first conductor, and a second conductor. The active conductor may be coupled to the active prong. The first conductor and second conductor may be coupled to the electrical circuit. The selector assembly may be actuated in the first position and complete an electrical loop between the first conductor and the active conductor. The selector assembly may be actuated in the second position and complete an electrical loop between the second conductor and the active conductor.

In aspects, the electrical circuit may be a resistor network.

In another aspect, the present disclosure provides a surgical system including an electrosurgical generator and an adaptor assembly. The electrosurgical generator is configured to provide electrosurgical energy. The adaptor assembly is in communication with the electrosurgical generator. The adaptor assembly includes a plug, a selector assembly, and a connector. The plug is coupled to the electrosurgical generator and is configured to receive the electrosurgical energy from the electrosurgical generator. The selector assembly includes a first position and a second position. The first position is configured to indicate a first setting of the electrosurgical generator, and the second position is configured to indicate a second setting of the electrosurgical generator. The selector assembly, when actuated, indicates the selected position to the electrosurgical generator to initiate the corresponding setting. The connector is configured to couple to a surgical instrument and convey the electrosurgical energy to the surgical instrument. The electrosurgical energy, when based on the first setting, is different from the electrosurgical energy, when based on the second setting.

In aspects, the electrosurgical generator receives an identification signal corresponding to an output voltage of an electrical circuit indicative of an identity of the adaptor assembly.

In aspects, the electrosurgical generator receives a first signal when the selected position is the first position and receives a second signal when the selected position is the second position.

In aspects, in response to receiving the first signal, the electrosurgical generator activates the first setting to provide the electrosurgical energy where the first setting corresponds to a first surgical mode, and in response to receiving the second signal, the electrosurgical generator activates the second setting to provide the electrosurgical energy where the second setting corresponds to a second surgical mode.

In aspects, the first surgical mode may be a vessel coagulation mode and the second surgical mode may be a vessel cutting mode.

In yet another aspect, the present disclosure provides a method in an electrosurgical system having an adaptor assembly coupled between an electrosurgical generator and a surgical instrument. The method includes receiving, by the electrosurgical generator, a first signal originating from the adaptor assembly indicative of a first setting of the electrosurgical generator. In response to receiving the first signal from the adaptor assembly, the method further includes providing an electrosurgical energy by the electrosurgical generator based on the first setting, receiving by the adaptor assembly the electrosurgical energy provided by the electrosurgical generator, conveying by the adaptor assembly the electrosurgical energy to the surgical instrument, and receiving by the surgical instrument the electrosurgical energy conveyed by the adaptor assembly.

In aspects, the method may further include receiving by the electrosurgical generator a second signal originating from the adaptor assembly indicative of a second setting of the electrosurgical generator and, in response to receiving the second signal from the adaptor assembly, providing electrosurgical energy by the electrosurgical generator based on the second setting.

In aspects, in providing electrosurgical energy by the electrosurgical generator based on the first setting, the first setting may correspond to a first surgical mode. In providing electrosurgical energy by the electrosurgical generator based on the second setting, the second setting may correspond to a second surgical mode.

In aspects, the first surgical mode may be a vessel coagulation mode and the second surgical mode may be a vessel cutting mode.

In aspects, the method may further include receiving, by the adaptor assembly, the electrosurgical energy from the electrosurgical generator, and receiving by the electrosurgical generator an identification signal originating from the adaptor assembly indicative of an identity of the adaptor assembly.

In aspects, the method may further include loading, by the generator, a first setting activated by a first signal, and a second setting activated by a second signal based on the identification signal.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a diagram of an exemplary electrosurgical system for switching between distinct modes and/or power settings, including a surgical instrument, generator, and an adaptor assembly, in accordance with aspects of the present disclosure;

FIG. 2 is a diagram of another exemplary electrosurgical system, with the adaptor assembly having an electrical circuit, in accordance with aspects of the present disclosure;

FIG. 3 is a flowchart of an exemplary method for switching between distinct modes and/or power settings, provided in accordance with aspects of the present disclosure; and

FIG. 4 is a flowchart of an exemplary method for determining the presence and identity of the electrosurgical instrument, provided in accordance with aspects of the present disclosure.

### DETAILED DESCRIPTION

Particular embodiments of the disclosure are described hereinbelow with reference to the accompanying drawings. In the following description, well-known functions or constructions may not be described in detail to avoid obscuring the present disclosure. As used herein, the term “distal” refers to that portion which is farther from the user while the term “proximal” refers to that portion which is closer to the user or surgeon.

The systems and methods of the disclosure detailed below may be incorporated into different types of surgical configurations or procedures. The particular illustrations and embodiments disclosed herein are merely exemplary and do not limit the scope or applicability of the disclosed technology.

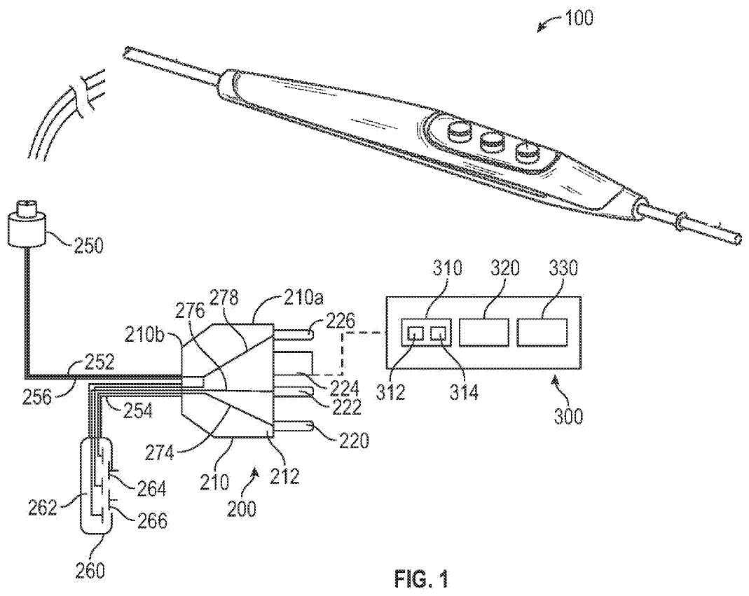

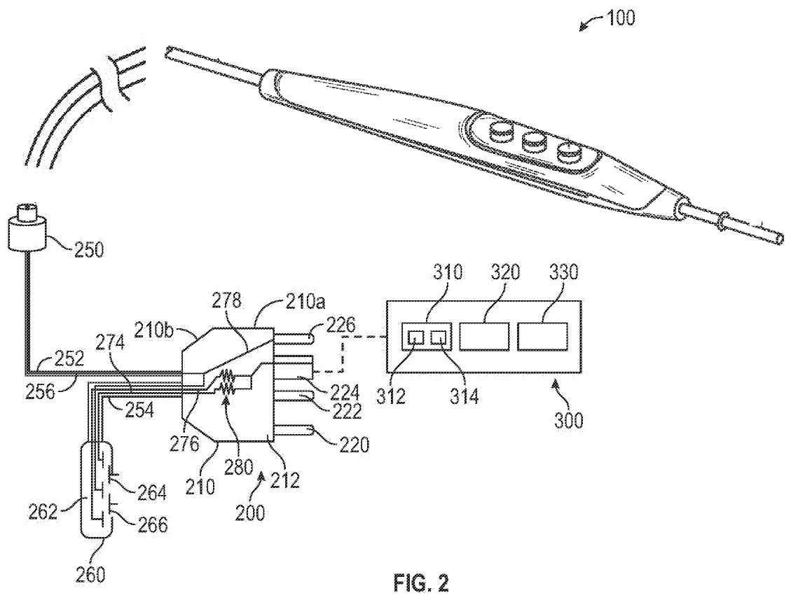

With reference to FIG. 1, a surgical system 1 is provided in accordance with the present disclosure. The surgical system 1 includes an energy-based surgical instrument, such as, for example, an electrosurgical instrument 100, coupled to a generator 300 via an adaptor assembly 200. The electrosurgical instrument 100 may be either monopolar type, as shown in FIG. 1, including one or more active electrodes (e.g., electrosurgical cutting probe, fulguration electrode(s), etc.) or a bipolar type (not shown), including one or more active and return electrodes (e.g., electrosurgical sealing forceps). The electrosurgical energy is supplied to the electrosurgical instrument 100 by the generator 300 via the adaptor assembly 200, allowing the electrosurgical instrument 100 to coagulate, cut, seal, fulgurate, and/or otherwise treat tissue. The surgical instrument illustrated herein is exemplary, and other suitable surgical instruments are also contemplated. For example, various types of energy may be provided by the generator to the instrument, such as, for example, providing ultrasonic energy to an ultrasonic instrument having an ultrasonic transducer. Aspects of the generator 300 and its components will be described in connection with FIG. 2.

With continuing reference to FIG. 1, the adaptor assembly 200 includes a plug 210, a connector 250, and a selector assembly 260. The plug 210 includes a plug housing 212 having a distal end portion 210a and a proximal end portion 210b. The distal end portion 210a includes a first prong 220, a second prong 222, a third prong 224, and an active prong 226. The ordering and shapes of the prongs illustrated in FIG. 1 is exemplary, and variations are contemplated. The proximal end portion 210b includes a connector cable 252 and a selector cable 254. The selector cable 254 is coupled to the proximal end portion 210b and extends proximally from the plug 210 to the selector assembly 260. In other embodiments, the selector assembly 260 may be coupled to the generator 300 wirelessly. The connector cable 252 is coupled to the proximal end portion 210b and extends proximally from the plug 210 to the connector 250. The distal end portion 210a of the plug 210 is configured to be inserted into a corresponding receptacle of the generator 300. The plug 210 is further configured to receive electrosurgical energy from the electrosurgical generator 300.

The selector assembly 260 includes a selector housing 262 and has a first button 264 and a second button 266 configured to be actuated by the user. The first button 264 and the second button 266 are disposed within the selector housing 262 of the selector assembly 260. The first button 264 is configured to correspond to a first setting of the generator 300, and the second button 266 is configured to correspond to a second setting of the generator 300 that is distinct from the first setting. In some embodiments, the selector assembly 260 may toggle/cycle through a set of preset settings through actuation of the first button 264 and/or the second button 266. In some embodiments, the selector assembly 260 may turn on and off a preset setting. In some embodiments, multiple actuation of the button of the selector assembly 260 may activate a preset setting. In some embodiments, actuation of any combination of the buttons of the selector assembly 260 may activate a preset setting or settings. The illustrated selector assembly is exemplary, and it is contemplated that the one or more buttons may instead be finger-actuated control buttons, rocker devices, a rotary dial, a slide selector, joystick, or any other suitable selection mechanism. In various embodiments, the selector assembly 260 may accommodate additional buttons or selection settings to allow for more than two setting selections. In various embodiments, a second recognized devices may be attached to generator 300 and configured to act as a tethered settings control. Additionally or alternatively, the selector assembly 260 may be coupled to, or integrated into the handle of the surgical instrument. As such, the selector assembly 260 may be utilized as a sterile selector assembly.

The adaptor assembly 200 further includes a first conductor 274, a second conductor 276 and an active conductor 278 disposed within the selector cable 254 and within the plug 210. The first conductor 274 is disposed within the selector housing 262 and extends distally to couple to the first prong 220 of the plug 210. The second conductor 276 is disposed within the selector housing 262 and extends distally to couple to the second prong. The active conductor 278 is disposed centrally between the first conductor 274 and the second conductor 276, and extends distally to couple to the active prong 226 of the plug 210. The first button 264 is disposed between the first conductor 274 and the active conductor 278 and is configured to complete an electrical loop upon actuation of the first button 264. The completed electrical loop between the first conductor 274 and the active conductor 278 generates a first signal. The second button 266 is disposed between the second conductor 276 and the active conductor 278 and is configured to complete an electrical loop upon actuation of the second button 266. The completed electrical loop between the second conductor 276 and the active conductor 278 generates a second signal. The positioning of the various conductors and the selector mechanism can vary from that described above. In various embodiments, the first signal is generated by a selector position that couples the active conductor 278 to the first conductor 274, and the second signal is generated by another selector position that couples the active conductor 278 to the second conductor 276. In various embodiments, the first signal and the second signal can have different electrical properties, such as different voltages. In various embodiments, the first signal and the second signal can have the same electrical properties or substantially the same electrical properties, but the signals are conveyed to different prongs of the plug 210.

The connector 250 is configured to couple to the electrosurgical instrument 100 via the connector cable 252. The connector cable 252 further includes at least one delivery conductor 256 disposed within the connector cable 252. In various embodiments, the connector cable 252 can include active and return conductors for a bipolar instrument. The delivery conductor 256 extends distally from the connector 250 towards the plug 210 and is coupled to the active conductor 278. The generator 300 conveys electrosurgical energy to the electrosurgical instrument 100 via the active conductor 278 and the delivery conductor 256 of the connector cable 252, based on the setting corresponding to the actuated first button 264 or the second button 266. It is contemplated that, the connector 250 may be any suitable configuration to accommodate connection to various types of the electrosurgical instruments 100, such as, for example monopolar, bipolar, or ultrasonic surgical instruments.

The disclosed embodiments are exemplary and variations are contemplated. For example, in the case of a monopolar instrument, instead of the connector 250 being coupled to the plug 210, the monopolar instrument is coupled to the generator 300 via a separate cable including a connector 250 coupled to an active prong 226 via connector cable 252. As another example, the instrument may be a monopolar electrosurgical device that has only one wire for the energy (activated via a footpedal) and that is connected to a “monopolar 1” port of a generator. The disclosed adaptor can connect to a “monopolar 2” port of the generator and can identify itself through the “monopolar 2” port. The adaptor button signals received by the “monopolar 2” port could cause the generator to cycle through settings and/or activate the energy out of the “monopolar 1” port. In various embodiments, one adaptor button can cause the generator to cycle through a set of preset settings, and another adaptor button can cause an activation that delivers energy to the instrument. In various embodiments, the user could still activate the instrument coupled to the “monopolar 1” port via a footpedal. Other variations are contemplated to be within the scope of the present disclosure.

With reference to FIG. 2, there is shown another embodiment of the adaptor assembly. In the illustrated embodiment, the adaptor assembly 200 includes an electrical circuit 280 disposed within the plug housing 212 and coupled to the third prong 224. The electrical circuit 280 is configured to deliver, to the generator 300, an identification signal 282 (e.g., an output voltage) indicative of an identity of the adaptor assembly 200. The identification signal 282 is derived based on electrosurgical energy delivered to the electrical circuit 280 via the active prong 226 from the generator 300. For example, a known voltage V_{REF} is applied to the electrical circuit 280 across resistors R1 and R2, and an output voltage of the electrical circuit 280 is provided by the voltage divider as, for example, V_{OUT}=V_{REF}*R1/(R1+R2). The generator 300 measures the output voltage V_{OUT} from the third prong 224 to determine the identity of the device coupled to the generator 300. In this case, the value of V_{OUT} identifies the device as an adaptor assembly 200. In accordance with aspects of the present disclosure, the generator 300 can respond to this identification by loading software routines that are configured to interpret signals received from the adaptor assembly 200 and initiate operations based on the received signals. Aspects of the generator 300 will be described in more detail later herein. The output voltage and identification signal 282 described above are exemplary, and other configurations for providing an identification signal 282 are contemplated to be within the scope of the present disclosure. In some embodiments, other methods of producing the identification signal 282 may be contemplated, such as for example, non-conducted methods including an RFID that allows the adaptor to provide its identity.

With continuing reference to FIG. 2, instead of the first conductor 274 and the second conductor 276 of the selector assembly 260 being coupled to the first prong 220 and the second prong 222, respectively, the first conductor 274 and the second conductor 276 may be coupled to the electrical circuit 280. The first button 264 is disposed between the first conductor 274 and the active conductor 278, and when actuated, completes an electrical loop through the first button 264. In various embodiments, the first button 264 can add an additional resistance to the voltage divider. A first signal is generated based on a known voltage delivered to the electrical circuit 280, and when the first button is actuated, and the output voltage of the electrical circuit 280 changes based on the additional resistance added by the first button 264. The second button 266 is disposed between the second conductor 276 and the active conductor 278, and when actuated, completes an electrical loop through the second button 266. In various embodiments, the second button 266 can add an additional resistance to the voltage divider. A second signal is generated based on a known voltage delivered to the electrical circuit 280, and when the second button is actuated, the output voltage of the electrical circuit 280 changes based on the additional resistance added by the second button 266. The electrical circuit 280 may be a resistor network, or any other suitable circuit for identification. The output voltage and the first and second signals described above are exemplary, and other configurations for providing signals corresponding to selector selections are contemplated to be within the scope of the present disclosure.

With reference to FIG. 1 and FIG. 2, the generator 300 generally includes a main controller 310, a high voltage DC power supply (HVPS) 320 (or other suitable power supply), an RF output stage 330 (or other suitable output circuitry depending on the energy to be delivered to electrosurgical instrument 100), one or more ports to accommodate the adaptor assembly 200 (not shown), and a user interface (not shown), e.g., a graphical user interface to enable the input and display of a variety of information such as modes of operation and power settings. In various embodiments, modes of operation and power settings may be configured by the user and stored on the generator 300. Additionally or alternatively, the selector assembly 260 may further include a memory and/or other logic configured to store the modes of operation and power settings configured by the user. Modes of operation may include, for example ablation, cutting, coagulation, sealing, pure cut, blend cut, valleylab, soft coagulation, fulgurate, spray, precise bipolar mode, standard bipolar mode, and macro bipolar mode. Power settings may be between zero and a power limit, such as, for example 30 W, 60 W, and 90 W, among others. Generator 300 is an exemplary embodiment, and it is contemplated that the generator may be any suitable device capable delivering energy to a surgical instrument.

The main controller 310 includes a microprocessor 312 connected to a computer-readable storage medium or memory 314, which may be a volatile type memory, e.g., RAM, or a non-volatile type memory, e.g., flash media, disk media, etc. The main controller 310 is coupled to the power supply 320 and/or the RF output stage 330, and the microprocessor 312 controls the output of energy from the generator 300 to the electrosurgical instrument 100. The microprocessor 312 is configured to receive signals from the adaptor assembly 200 and to control the output of energy from the generator 300 to the electrosurgical instrument 100 based on the mode of operation and power setting indicated by the signals received from the adaptor assembly 200. The memory 314 may store suitable instructions, to be executed by the microprocessor 312, for detecting the button selection of the selector assembly 260, and determining corresponding actions based on the button selection of the selector assembly 260. In various embodiments, the generator's operation corresponding to each button selection of the selector assembly 260 can be stored in the memory 314 and may be configured be based on or updated according to user inputted settings on the generator 300 for each button selection of the selector assembly 260. In various embodiments, instructions stored in the memory 314 may be implemented via one or more software applications executed on a processor.

In operation, the electrosurgical instrument 100 is coupled to the connector 250, and the plug 210 of the adaptor assembly 200 is inserted into the generator 300. The memory 314 loads instructions for detecting button selection of the selector assembly 260 and loads instructions for operations to be performed based on the button selection of the selector assembly 260. The generator 300 may, for example, respond to a first button 264 actuation by activating settings for coagulation at 30 W and respond to a second button 266 actuation by activating settings for coagulation at 60 W, or respond to a first button 264 actuation by activating macro mode and settings for dissecting, and responding to a second button 266 actuation by activating standard mode with low power settings for coagulation. These generator responses are exemplary, and other settings and operations are contemplated to be within the scope of the present disclosure.

As the first button 264 of the selector assembly 260 is actuated, the electrical loop between the first conductor 274 and the active conductor 278 is completed and the first signal is delivered to the generator 300. The microprocessor 312 receives the first signal and loads the corresponding actions from the memory 314. The microprocessor 312 activates the generator 300 by changing the mode of operation and/or power setting of generator 300, among other possible settings. The generator 300 provides electrosurgical energy through the active prong 226 of the adaptor assembly 200 to the electrosurgical instrument 100 based on the mode of operation and/or power setting of the generator 300. The surgical procedure is performed with the electrosurgical instrument 100 in the desired mode of operation and/or power setting.

During the surgical procedure, when the second button 266 of the selector assembly 260 is actuated, the electrical loop between the first conductor 274 and the active conductor 278 is released, and the electrical loop between the second conductor 276 and the active conductor 278 is completed. The second signal is conveyed to the generator 300 and is received by the microprocessor 312. The microprocessor 312 loads the corresponding actions from the memory 314 and activates the generator 300 by changing the mode of operation and/or power setting of generator 300, among other possible settings. The generator 300 provides electrosurgical energy through the active prong 226 of the adaptor assembly 200 to the electrosurgical instrument 100 based on the mode of operation and/or power setting of the generator 300. It is contemplated, that the user may rapidly and frequently change between the buttons on the selector assembly 260 throughout the surgical procedure, and thereby provide surgical flow and efficiency.

In some embodiments, prior to actuation of the buttons of the adaptor assembly 200, electrosurgical energy is delivered from the generator 300 to the adaptor assembly 200. The output voltage of the electrical circuit 280 is measured to determine the presence and identity of the adaptor assembly 200. The identification signal 282 is received by the microprocessor 312 of the generator 300 and the identity and presence of the adaptor assembly 200 is determined by the generator 300.

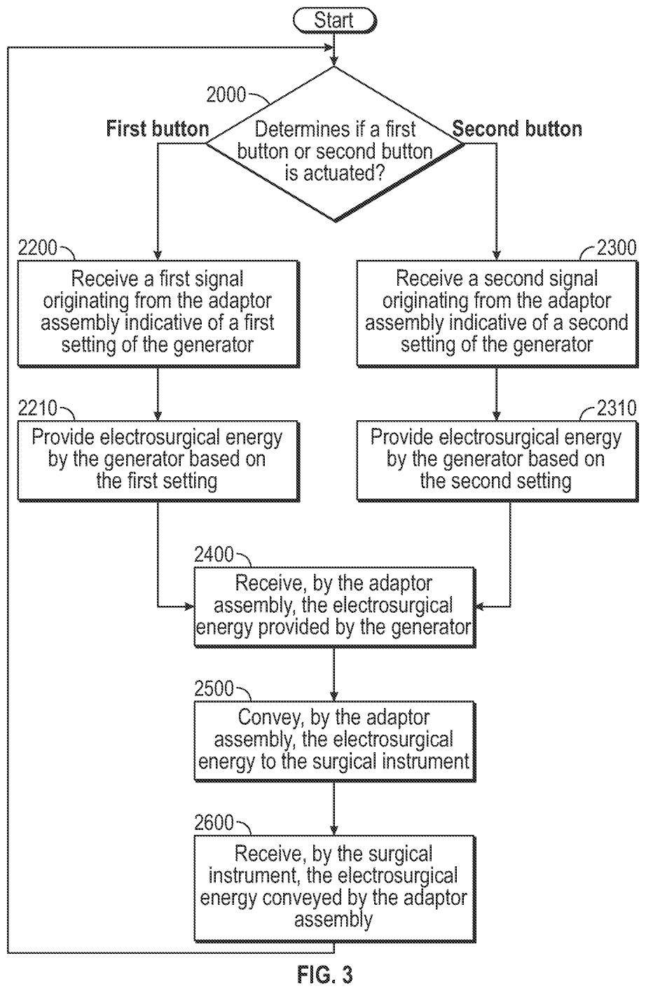

With reference to FIG. 3, a flowchart is provided showing the surgical system 1 switching between distinct modes and/or power settings. At step 2000, the adaptor assembly 200 determines whether a first button 264 or second button 266 is actuated.

At step 2200, if the first button 264 is actuated, the generator 300 receives a first signal originating from the adaptor assembly 200 indicative of a first setting of the generator 300. In response to receiving the first signal from the adaptor assembly 200, at step 2210, electrosurgical energy is provided by the generator 300 in accordance with the first setting. In an exemplary embodiment, at step 2210, providing electrosurgical energy by generator 300, in accordance with the first setting, corresponds to a first surgical mode having settings suitable for vessel coagulation. In embodiments, the first surgical mode may be other surgical modes, such as, for example tissue cutting mode, tissue coagulation mode, resection mode, or sealing mode.

At step 2300, if the second button 266 is actuated, the generator 300 receives a second signal originating from the adaptor assembly 200 indicative of a second setting of the generator 300. In response to receiving the second signal from the adaptor assembly 200, at step 2310, electrosurgical energy is provided by the generator 300 in accordance with the second setting. In an exemplary embodiment, at step 2310, providing electrosurgical energy by generator 300, in accordance with the second setting, corresponds to a second surgical mode having settings suitable for vessel cutting. In aspects, the second surgical mode may be other surgical modes, such as, for example tissue cutting mode, tissue coagulation mode, resection mode, or sealing mode.

The adaptor assembly 200, at step 2400, receives the electrosurgical energy provided by the generator 300. At step 2500, the adaptor assembly 200 conveys the electrosurgical energy to the electrosurgical instrument 100, and the electrosurgical instrument 100 receives the electrosurgical energy, at step 2600. Until the surgical procedure is complete, the generator may switch between distinct modes and/or power settings according to the selection of the first button 264 or second button 266, at step 2000.

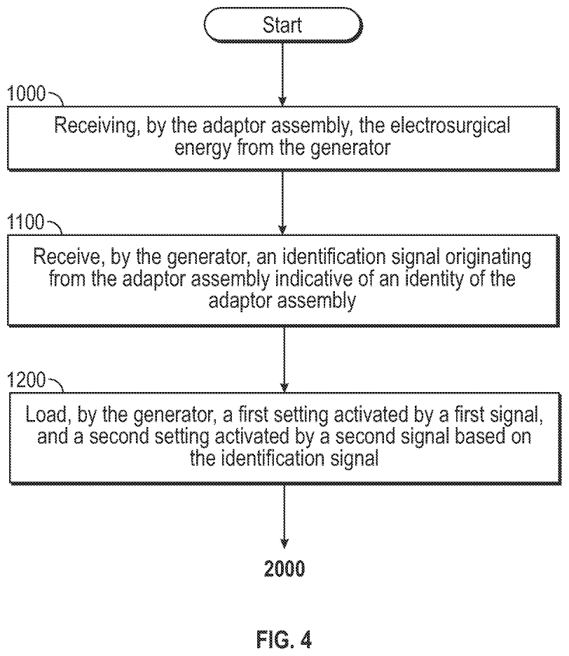

With reference to FIG. 4, a flowchart is provided showing the surgical system 1 determining the presence and identity of the adaptor assembly 200 prior to switching between distinct modes and/or power settings via the adaptor assembly 200, and after insertion of the adaptor assembly into generator 300. At step 1000, the adaptor assembly 200 receives electrosurgical energy from generator 300. At step 1100, an identification signal originating from the adaptor assembly indicative of an identity of the adaptor assembly 200 is received. At step 1200, the generator loads a first setting and a second setting stored by the generator 300 according to the identification signal.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An adaptor assembly comprising:
a plug configured to couple to an electrosurgical generator and receive electrosurgical energy from the electrosurgical generator;
a selector assembly including a first position configured to indicate a first setting of the electrosurgical generator and a second position configured to indicate a second setting of the electrosurgical generator, wherein when actuated, the selector assembly indicates the selected position to the electrosurgical generator to initiate the corresponding setting; and
a connector configured to couple to a surgical instrument and convey the electrosurgical energy to the surgical instrument,
wherein the electrosurgical energy, when based on the first setting, is different from the electrosurgical energy, when based on the second setting.

2. The adaptor assembly of claim 1, wherein the plug further includes a first prong, a second prong, a third prong, and an active prong, wherein each prong is configured to be inserted into a corresponding receptacle of the electrosurgical generator.

3. The adaptor assembly of claim 2, further comprising an active conductor coupled to the active prong, wherein when the selector assembly is actuated in the first position or the second position, the electrosurgical energy is conveyed from the active prong through the active conductor to the surgical instrument.

4. The adaptor assembly of claim 2, further comprising a first conductor coupled to the first prong, wherein when the selector assembly is actuated in the first position, the selector assembly completes an electrical loop between the first conductor and the active conductor.

5. The adaptor assembly of claim 4, further comprising a second conductor coupled to the second prong, wherein when the selector assembly is actuated in the second position, the selector assembly completes an electrical loop between the second conductor and the active conductor.

6. The adaptor assembly of claim 2, further comprising an electrical circuit coupled to the third prong.

7. The adaptor assembly of claim 6, wherein the electrical circuit is configured to deliver an output voltage indicative of an identity of the adaptor assembly when the adaptor assembly is coupled to the electrosurgical generator, the output voltage being derived from at least a portion of the electrosurgical energy from the electrosurgical generator.

8. The adaptor assembly of claim 6, further comprising:
an active conductor coupled to the active prong; and
a first conductor and a second conductor coupled to the electrical circuit,
wherein when the selector assembly is actuated in the first position, the selector assembly completes an electrical loop between the first conductor and the active conductor, and
wherein when the selector assembly is actuated in the second position, the selector assembly completes an electrical loop between the second conductor and the active conductor.

9. The adaptor assembly of claim 6, wherein the electrical circuit is a resistor network.

10. A surgical system comprising:
an electrosurgical generator configured to provide electrosurgical energy; and
an adaptor assembly in communications with the electrosurgical generator, the adaptor assembly including:
a plug coupled to the electrosurgical generator and configured to receive the electrosurgical energy from the electrosurgical generator;
a selector assembly including a first position configured to indicate a first setting of the electrosurgical generator and a second position configured to indicate a second setting of the electrosurgical generator, wherein when actuated, the selector assembly indicates the selected position to the electrosurgical generator to initiate the corresponding setting; and
a connector configured to couple to a surgical instrument and convey the electrosurgical energy to the surgical instrument,
wherein the electrosurgical energy, when based on the first setting, is different from the electrosurgical energy, when based on the second setting.

11. The surgical system of claim 10, wherein the electrosurgical generator receives an identification signal corresponding to an output voltage of an electrical circuit indicative of an identity of the adaptor assembly.

12. The surgical system of claim 10, wherein the electrosurgical generator receives a first signal when the selected position is the first position and receives a second signal when the selected position is the second position.

13. The surgical system of claim 12, wherein in response to receiving the first signal, the electrosurgical generator activates the first setting to provide the electrosurgical energy, the first setting corresponding to a first surgical mode, and in response to receiving the second signal, the electrosurgical generator activates the second setting to provide the electrosurgical energy, the second setting corresponding to a second surgical mode.

14. The surgical system of claim 13, wherein the first surgical mode is a vessel coagulation mode and the second surgical mode is a vessel cutting mode.

15. A method in an electrosurgical system having an adaptor assembly coupled between an electrosurgical generator and a surgical instrument, the method comprising:
receiving, by the electrosurgical generator, a first signal originating from the adaptor assembly indicative of a first setting of the electrosurgical generator;
in response to receiving the first signal from the adaptor assembly, providing electrosurgical energy by the electrosurgical generator based on the first setting;
receiving, by the adaptor assembly, the electrosurgical energy provided by the electrosurgical generator; and
conveying, by the adaptor assembly, the electrosurgical energy to the surgical instrument; and
receiving, by the surgical instrument, the electrosurgical energy conveyed by the adaptor assembly.

16. The method of claim 15, further comprising:
receiving, by the electrosurgical generator, a second signal originating from the adaptor assembly indicative of a second setting of the electrosurgical generator; and
in response to receiving the second signal from the adaptor assembly, providing electrosurgical energy by the electrosurgical generator based on the second setting.

17. The method of claim 16, wherein in providing electrosurgical energy by the electrosurgical generator based on the first setting, the first setting corresponds to a first surgical mode, and in providing electrosurgical energy by the electrosurgical generator based on the second setting, the second setting corresponds to a second surgical mode.

18. The method of claim 17, wherein the first surgical mode is a vessel coagulation mode and the second surgical mode is a vessel cutting mode.

19. The method of claim of claim 15, further comprising:
receiving, by the adaptor assembly, the electrosurgical energy from the electrosurgical generator; and
receiving, by the electrosurgical generator, an identification signal originating from the adaptor assembly indicative of an identity of the adaptor assembly.

20. The method of claim of claim 19, further comprising loading, by the generator, a first setting activated by a first signal, and a second setting activated by a second signal based on the identification signal.
